(19) United States
(12) Patent Application Publication
(10) Pub. No.: US 2020/0078841 A1
(43) Pub. Date: Mar. 12, 2020

(54) MICROBIAL SOIL ENHANCEMENTS

(71) Applicant: Khanh Le, San Jose, CA (US)

(72) Inventor: Khanh Le, San Jose, CA (US)

(21) Appl. No.: 16/542,154

(22) Filed: Aug. 15, 2019

Related U.S. Application Data
(62) Division of application No. 15/499,137, filed on Apr. 27, 2017.

(51) Int. Cl. B09C 1/10; C12N 1/20; C12M 1/16; C02F 3/34; C05F 11/08; C05G 3/00; A01N 63/00

(52) U.S. Cl. CPC B09C 1/10; C12N 1/20; C12M 1/16; C02F 3/34; A01C 3/00; C05G 3/0047; A01N 63/00; C05G 3/00; C05F 11/08

(57) ABSTRACT

A method for forming soil enhancement includes forming microbes in a concentrated form of at least 1×10⁷ cfu/ml (colony-forming units per milliliter); and dry forming the microbes onto humic acid.

### BACKGROUND

The present invention relates to microbial enhancements for soil.

Countries such as the U.S., Canada, and Mexico are the major producers of fruit & vegetable crops. Rise in organic & environment-friendly farming practices has increased the demand for agricultural microbials, especially in the U.S. While natural soil enhancements are preferable, chemical fertilizers are still the dominant mode of helping farmers grow. The main environmental problem associated with fertilizer use is contamination of water with nitrates and phosphates. The nitrogen from fertilizers and manures are eventually converted by bacteria in the soil to nitrates.

Bacterial agricultural microbials are helpful to the crops in a way that they detoxify the soil and fight the root diseases and provide stability to the soil system. They help in nitrogen fixation, phosphate solubilization, iron sequestration, and phytohormone level modulation in crops. Due to these factors, the bacterial segment dominates the agricultural microbials market.

### SUMMARY OF THE INVENTION

In one aspect, a method for forming soil enhancement includes forming microbes in a concentrated form of at least 1×10⁷ cfu/ml (colony-forming units per milliliter); and dry forming the microbes onto humic acid.

The dry forming operation includes either dry-spraying or freeze drying with lyophilization. The spray drying of the microbe on to humic acid granular can be done, or alternatively, the microbes are first freeze dried using a method of lyophilization. Then the freeze-dried microbes are blended to dry humic, and other carriers. Specific microbes trait and profile first are grown in a lab using nutrient agar plate. Then lyophilization process is used to dry the combination.

In another aspect, a method for forming soil enhancement includes forming microbes in a concentrated form of at least 1×10⁷ cfu/ml (colony-forming units per milliliter); dry spraying the microbes onto a granular blend of humic acid and urea.

In another aspect, a method for forming soil enhancement includes forming microbes in a concentrated form of at least 1×10⁷ cfu/ml (colony-forming units per milliliter); dry spraying the microbes onto a granular blend of humic acid and kelp or seaweed.

In another aspect, a method for forming soil enhancement includes forming microbes in a concentrated form of at least 1×10⁷ cfu/ml (colony-forming units per milliliter); dry spraying the microbes onto a granular blend of humic acid and waste. The waste can be fish meal, compost, or manure (such as mammal manure, animal manure, bird manure, chicken manure, among others).

In a further aspect, a method for forming soil enhancement includes forming microbes in a concentrated form of at least 1×10⁷ cfu/ml (colony-forming units per milliliter); dry spraying the microbes onto a granular blend of humic acid and one or more of: nitrogen, phosphorous, and potassium (NPK).

In another aspect, a method enhances soil by preparing a microbial solution with microbes, a growth medium, and water; iteratively and selectively breeding generations of microbes to arrive at a predetermined microbial solution in a concentrated form of at least 1×10⁷ cfu/ml (colony-forming units per milliliter); dry spraying humic acid to cover urea granular blend(s); and depositing the microbial solution onto the humic acid for enriching the soil with micronutrients, microbial cultures and organic materials.

In another aspect, an apparatus for enhancing soil includes a tank for a microbial solution with microbes, a growth medium, and water; a sequencer to iteratively and selectively breeding generations of microbes to arrive at a predetermined microbial solution in a highly concentrated form of at least Ix 10⁷ cfu/ml (colony-forming units per milliliter); a dry sprayer to spray humic acid to cover urea granular blend(s); and a unit for depositing the microbial solution onto the humic acid to enrich the soil with micronutrients, microbial cultures and organic materials.

Implementations of the above aspects may include one or more of the following. The microbes can be selected from Bacillus (B.) acidiceler, B. acidicola, B. acidiproducens, B. acidocaldarius, B. acidoterrestrisr, B. aeolius, B. aerius, B. aerophilus, B. agaradhaerens, B. agri, B. aidingensis, B. akibai, B. alcalophilus, B. algicola, B. alginolyticus, B. alkalidiazotrophicus, B. alkalinitrilicus, B. alkalisediminis, B. alkalitelluris, B. altitudinis, B. alveayuensis, B. alvei, B. amyloliquefaciens, B. a. subsp. Amyl, aoliquefaciens, B. a. subsp. plantarum, B. amylolyticus, B. andreesenii, B. aneurinilyticus, B. anthracis, B. aquimaris, B. arenosi, B. arseniciselenatis, B. arsenicus, B. aurantiacus, B. arvi, B. aryabhattai, B. asahii, B. atrophaeus, B. axarquiensis, B. azotofixans, B. azotoformans, B. badius, B. barbaricus, B. bataviensis, B. beijingensis, B. benzoevorans, B. beringensis, B. berkeleyi, B. beveridgei, B. bogoriensis, B. boroniphilus, B. borstelensis, B. brevis Migula, B. butanolivorans, B. canaveralius, B. carboniphilus, B. cecembensis, B. cellulosilyticus, B. centrosporus, B. cereus, B. chagannorensis, B. chitinolyticus, B. chondroitinus, B. choshinensis, B. chungangensis, B. cibi, B. circulans, B. clarkii, B. clausii, B. coagulans, B. coahuilensis, B. cohnii, B. composti, B. curdlanolyticus, B. cycloheptanicus, B. cytotoxicus, B. daliensis, B. decisifrondis, B. decolorationis, B. deserti, B. dipsosauri, B. drentensis, B. edaphicus, B. ehimensis, B. eiseniae, B. enclensis, B. endophyticus, B. endoradicis, B. farraginis, B. fastidiosus, B. fengqiuensis, B. firmus, B. flexus, B. foraminis, B. fordii, B. formosus, B. fortis, B. fumarioli, B. funiculus, B. fusiformis, B. galactophilus, B. galactosidilyticus, B. galliciensis, B. gelatini, B. gibsonii, B. ginsengi, B. ginsengihumi, B. ginsengisoli, B. globisporus, B. g. subsp. globisporus, B. g. subsp. marinus, B. glucanolyticus, B. gordonae, B. gottheilii, B. graminis, B. halmapalus, B. haloalkaliphilus, B. halochares, B. halodenitrificans, B. halodurans, B. halophilus, B. halosaccharovorans, B. hemicellulosilyticus, B. hemicentroti, B. herbersteinensis, B. horikoshii, B. horneckiae, B. horti, B. huizhouensis, B. humi, B. hwajinpoensis, B. idriensis, B. indicus, B. infantis, B. infernus, B. insolitus, B. invictae, B. iranensis, B. isabeliae, B. isronensis, B. jeotgali, B. kaustophilus, B. kobensis, B. kochii, B. kokeshiiformis, B. koreensis, B. korlensis, B. kribbensis, B. krulwichiae, B. laevolacticus, B. larvae, B. laterosporus, B. lautus, B. lehensis, B. lentimorbus, B. lentus, B. licheniformis, B. ligniniphilus, B. litoralis, B. locisalis, B. luciferensis, B. luteolus, B. luteus, B. macauensis, B. macerans, B. macquariensis, B. macyae, B. malacitensis, B. mannanilyticus, B. marisflavi, B. marismortui, B. marmarensis, B. massiliensis, B. megaterium, B. mesonae, B. methanolicus, B. methylotrophicus, B. migulanus, B. mojavensis, B. mucilaginosus, B. muralis, B. murimartini, B. mycoides, B. naganoensis, B. nanhaiensis, B. nanhaiisediminis, B. nealsonii, B. neidei, B. neizhouensis, B. niabensis, B. niacini, B. novalis, B. oceanisediminis, B. odysseyi, B. okhensis, B. okuhidensis, B. oleronius, B. oryzaecorticis, B. oshimensis, B. pabuli, B. pakistanensis, B. pallidus, B. pallidus, B. panacisoli, B. panaciterrae, B. pantothenticus, B. parabrevis, B. paraflexus, B. pasteurii, B. patagoniensis, B. peoriae, B. persepolensis, B. persicus, B. pervagus, B. plakortidis, B. pocheonensis, B. polygoni, B. polymyxa, B. popilliae, B. pseudalcalophilus, B. pseudofirmus, B. pseudomycoides, B. psychrodurans, B. psychrophilus, B. psychrosaccharolyticus, B. psychrotolerans, B. pulvifaciens, B. pumilus, B. purgationiresistens, B. pycnus, B. qingdaonensis, B. qingshengii, B. reuszeri, B. rhizosphaerae, B. rigui, B. ruris, B. safensis, B. salarius, B. salexigens, B. saliphilus, B. schlegelii, B. sediminis, B. selenatarsenatis, B. selenitireducens, B. seohaeanensis, B. shacheensis, B. shackletonii, B. siamensis, B. silvestris, B. simplex, B. siralis, B. smithii, B. soli, B. solimangrovi, B. solisalsi, B. songklensis, B. sonorensis, B. sphaericus, B. sporothermodurans, B. stearothermophilus, B. stratosphericus, B. subterraneus, B. subtilis, B. s. subsp. inaquosorum, B. s. subsp. spizizenii, B. s. subsp. subtilis, B. taeanensis, B. tequilensis, B. thermantarcticus, B. thermoaerophilus, B. thermoamylovorans, B. thermocatenulatus, B. thermocloacae, B. thermocopriae, B. thermodenitrificans, B. thermoglucosidasius, B. thermolactis, B. thermoleovorans, B. thermophilus, B. thermoruber, B. thermosphaericus, B. thiaminolyticus, B. thioparans, B. thuringiensis, B. tianshenii, B. trypoxylicola, B. tusciae, B. validus, B. vallismortis, B. vedderi, B. velezensis, B. vietnamensis, B. vireti, B. vulcani, B. wakoensis, B. weihenstephanensis, B. xiamenensis, B. xiaoxiensis, and B. zhanjiangensis. With a member of Bacillus as the microbe, the process can use a carrier from one of: liquid, water, dry humic acid, wet humic acid, urea, soil wetting aid or a penetrant.

The microbes can be: Bacillus amyloliquefaciens at 5.85×107\hat{ }7 cfu/ml, Bacillus lichniformis at 1.80×107\hat{ }7 cfu/ml, Bacillus pumilus at 4.05×107\hat{ }7 cfu/ml, or Bacillus subtilis at 6.30×107\hat{ }7 cfu/ml. Humic substances or leonardite and urea and water can be used with the microbes. Polyloxy-(1,2-Ethanedily), Alpha-(nonylphenyl)-omega-hydroxy can be used with the microbes. The solution can also include humic substances or leonardite and water. The microbial solutions can be applied through spraying, wetting, dipping, misting, drenching, showering, fogging, soaking, dampening, drizzling, dousing and splashing.

Advantages of the solutions may include one or more of the following. Soil enrichment solutions of the system stimulate plant growth, rejuvenate the soil, and promote the growth of beneficial soil microorganisms. Some embodiments also provide natural pathogens for the prevention, control and/or cure of turf and plant diseases and other purposes encouraging germination and/or growth. The solutions contain microorganism spores and/or colonies that remain viable for at least about a year when stored at room temperature. The solutions provide soil enrichment solutions containing viable microorganism spores and/or colonies, particularly those useful for enriching poor, disturbed soils or soils having little or no microbial activity because of the heavy past use of chemicals and/or fertilizers. The systems provide solutions containing viable micro organism spores and/or colonies of beneficial fungicides that can be used for seed, turf, and leaf treatment for the prevention, control, and/or cure of turf and plant diseases and other beneficial purposes. The solutions also provide soil enrichment solutions containing microorganism spores and/or colonies that remain at least about 90% viable for up to at least about 12, preferably 18 months at room temperature, i.e., about 20° to 25° C.

These and other advantages are achieved by the present invention, which provides a method of preserving and solutions containing microbial spores and/or colonies.

### BRIEF DESCRIPTION OF THE FIGURES

FIG. 1 shows an exemplary process to selectively breed the microbes for agricultural use and incorporate the microbes into urea.

FIGS. 2A-2B show exemplary processes to produce the microbial products.

FIG. 2C shows a system to produce the microbial products.

FIGS. 3A-3B show exemplary antifungal activity express by different Bacillus spp. strains.

FIG. 4 shows exemplary cellulolytic enzymes synthesized by the biological control agent which can be involved in two plant defense mechanism against phyto-pathogenic fungi.

FIG. 5 shows exemplary soil enhancement enzyme profiles isolation standards.

FIGS. 6A-6B show exemplary systems for dry spraying humic acid to cover urea granular blend(s) and and depositing the microbial solution onto the humic acid covering the urea granular blend(s).

FIG. 7 shows an exemplary system to selectively breed the microbes.

### DETAILED DESCRIPTION OF THE INVENTION

A selectively bred microbial solution is disclosed with multiple single microbial series separately cultivated and followed with cross cultivation among those microbial series in a specific sequence and contains each of those microbial series, and by-products produced by those crossly cultivated microbial series are used for applications in modifying soil quality, activating soil, effectively degrading soil pollution, and helping growth of crops in a soil enhancement embodiment. After the selective breeding through the fermentation, the selectively bred naturally-occurring microorganisms have the ability to penetrate through the soil while enriching with micronutrients, microbial cultures and organic materials in a highly concentrated stage.

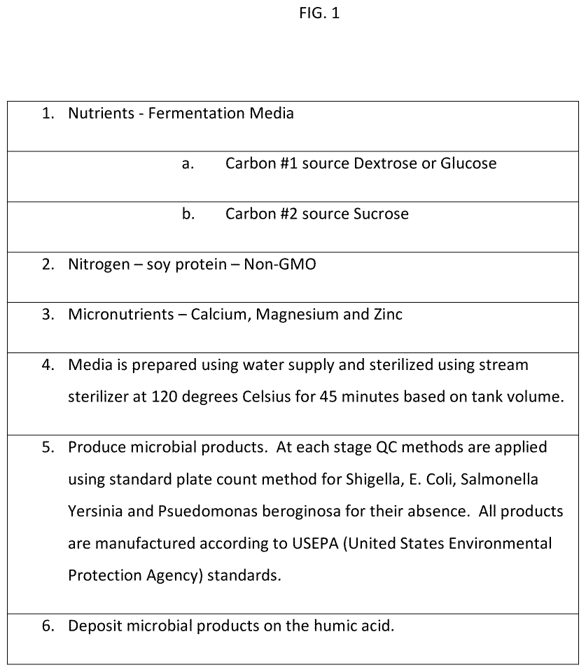

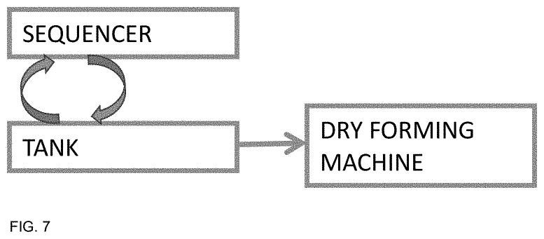

FIG. 1 shows an exemplary process to selectively breed the microbes for agricultural use and FIG. 7 shows an exemplary system to selectively breed the microbes. First, fermentation media are prepared with a nutrient supply (1). The nutrients can include a carbon source, Dextrose, Glucose, or maltodextrin. Additional carbon sources can be used with the dextrose or glucose singly or in combination. For example, another carbon source can be sucrose, for example. Next, a nitrogen source is provide such as soy protein that has not been genetically modified (2). Next, in (3), micronutrients—Calcium, Magnesium and Zinc are provided. A person of ordinary skilled in the art appreciates that various compositions of the fermentation media can be prepared so long as the nutrients, one or more of the carbon sources, and the micronutrients are included.

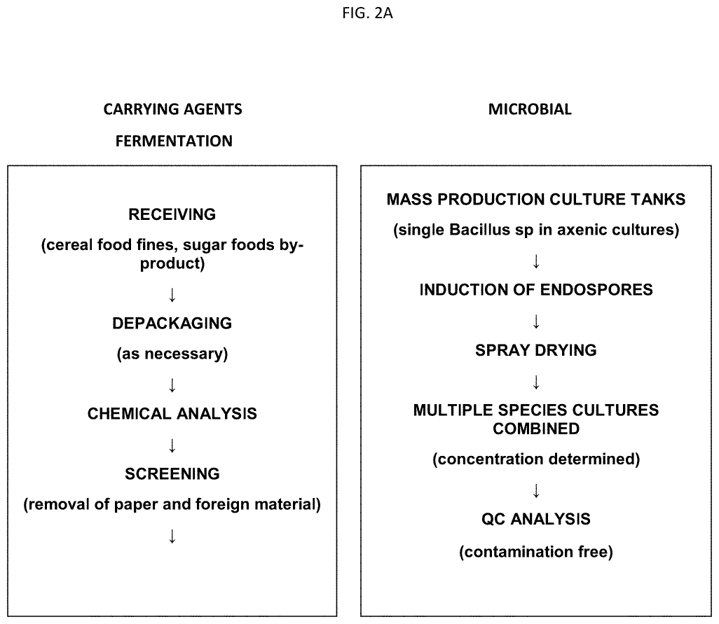

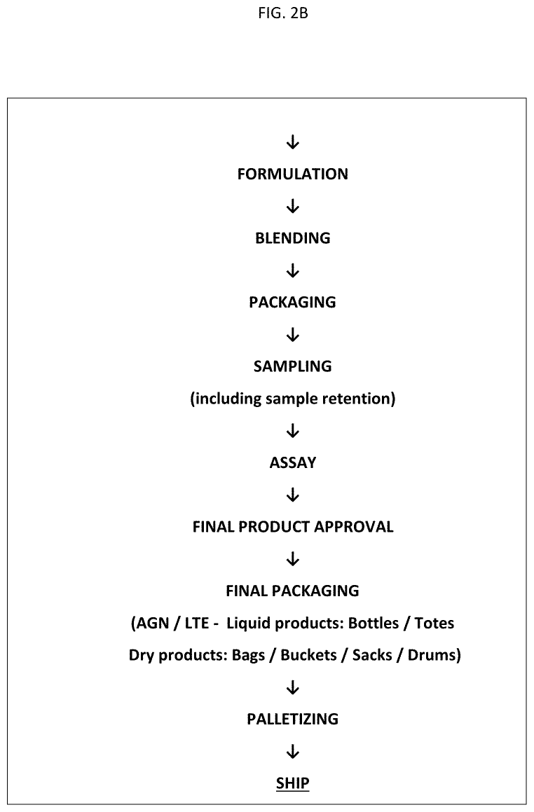

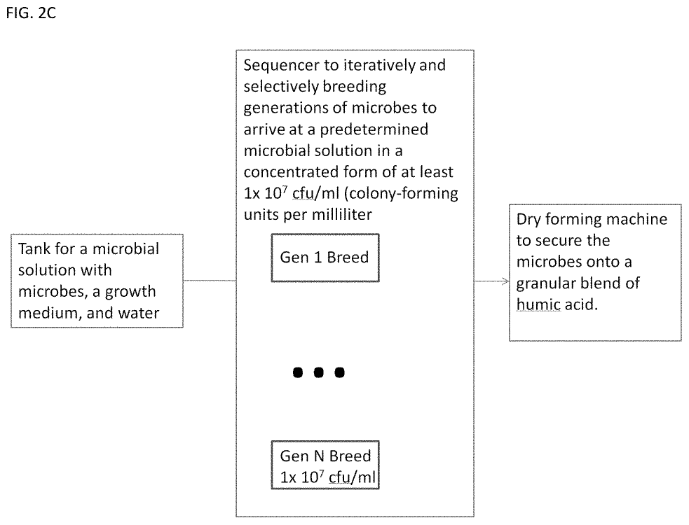

In (4), the fermentation media is prepared using water supply and sterilized using stream sterilizer at 120 degrees Celsius for 45 minutes, but the temperature and time can be varied in accordance with tank volume. In (5), the process produces the microbial products, as is detailed in FIGS. 2A-2B. At each stage, quality control methods are applied using standard plate count method for Shigella, E. Coli, Salmonella Yersinia and Psuedomonas beroginosa for their absence. All products are manufactured according to USEPA (United States Environmental Protection Agency) standards.

In (6), a dry forming machine provides a coating of microbes on humic acid. The dry forming operation includes either dry-spraying or freeze drying with lyophilization. The spray drying of the microbe on to humic acid granular can be done, or alternatively, the microbes are first freeze dried using a method of lyophilization. Then the freezed dried microbes are blended to dry humic, and other carriers. Specific microbes trait and profile first are grown in a lab using nutrient agar plate. Then lyophilization process is used to dry the combination.

The microbes can be: Bacillus amyloliquefaciens at 7.5×10\hat{ }8 cfu/ml, Bacillus lichniformis at 7.5×107\hat{ }7 cfu/ml, Bacillus pumilus at 2.5×10\hat{ }8 cfu/ml, or Bacillus subtilis at 1×10\hat{ }8 cfu/ml, or Bacillus megaterium at 2.5×10\hat{ }8 cfu/ml.

The Microbial Strain selection and profile of microbial genes are carefully selected to form the formulation of products. Through strain selections, screening and improvement, the system generates various bio-fertilizer products for rejuvenating soil and promote plant growth. For example, Bacillus subtilus has 4,100 genes. These genes each contain approximately 2000 traits. Each one of these traits and its mutation has over 1000 profile and sub-profile.

With a member of Bacillus as the microbe, the process can include a carrier from one of: liquid, water, dry humic acid, wet humic acid, urea, NPK, or soil wetting aid. When applied in the field to plants, billions of the selectively bred bacteria operate to convert and breakdown organic matter into a form of micronutrient for plant uptake. The microbial solution can be applied through spraying, wetting, dipping, misting, drenching, showering, fogging, soaking, dampening, drizzling, dousing and splashing.

The biodiversity of Bacillus group and beneficial traits of bacillus species are useful in plant protection. Bacillus genus is widely spread in nature. Bacillus species such as B. Subtilus, B. Megaterium, B. Amyloliquefaciens, B. lichniformis are carefully selected, for their specific profile which contains beneficial traits for plant protection and growth promotion that comprise the synthesis in broad-spectrum with active metabolites and easily adaptation in various environment conditions that benefits plant bacterial interaction and advantageous of formulation process.

As plants roots exudates and lysates attracts and stimulate microbial activity in the root surrounding soil, the zhizosphere (chemical space around the roots) became highly populated. Beneficial Bacillus spp. strains can compete with other bacteria and fungi that could adversely affect crops. They can inhibit phytopathogenic attacks such as “Basal Stem Rot, Phytophthora, Fusarium”, or induce host-plant defense system against potential pathogenic attacks, stimulate plant growth, improve nutrient uptake, and reduce negative environment traits.

Beneficial traits with agricultural purpose in Bacillus Subtilis and related species are detailed next. The species of bacillus group, particularly B. Subtilus, B. Megaterium, B. Amyloliquefaciens, B. lichniformis are extremely importance in agriculture, as phytopathogenic antagonist or plant growth promoters. It is often referring as “Plant Growth Promoting rhizobacteria” or PGPR. PGPR are naturally occurring soil bacteria that have the ability to colonize the roots, and the high concentration and the amount of bacteria artificially created (added) as detailed above enhances the stimulation of plant growth by phytohormones production or by releasing beneficial organic compounds.

Beside plant growth stimulation, Bacillus Subtilis and its related species strain are involved in plant protection against phyto-pathogenic attacks. They act directly against pathogens by producing extracellular lytic enzyme and secondary metabolites with inhibitory growth action or interfere by quorum quenching to disturb cell-to-cell communication of the infectious expression in pathogenic bacteria. They could also compete with plant pathogen for the available nutrient and niche. Another important role is the reduction of the infection process by inducing defense response in the host plant.

Each single microbial series is separately cultivated in its designated cultivation medium, and the optimal pH in the growing and reproduction of different microbial series also varies. Therefore, proper control and regulation of pH of the cultivation medium are provided in the course of bacterial cultivation and fermentation. The microbial series acquires energy through aerobic respiration. However, the aerobic respiration generally has to rely upon only the oxygen dissolved in the cultivation medium, i.e., the dissolved oxygen, and the containment of the dissolved oxygen in the cultivation medium is not always provided in sufficient amount and will be soonest consumed by bacteria since oxygen is difficult to get dissolved in water. Therefore, constant air supply to the microbial series is provided without interruption in the course of the cultivation and fermentation of the microbial series. Compositions of cultivation medium selected and the optimal growing environment conditions for each microbial series are detailed as follows:

When the cultivation of each microbial series is saturated in its cultivation medium, a cross cultivation is followed. The compound microbial preparation differs from a single bacteria species or a single microbial product for soil modification. In some embodiments, the microbial life activities from multiple preselected microbial series are provided that are mutually coordinated and contained for crops or plants to get the results of specific fertilizers; that is, multiple microorganisms are screened from the soil and selectively bred to become capable of improving nutrition of the crops, and then to provide nitrogen, phosphor, and potassium fertilizers important to the growth of the plants in organic means by taking advantage of interaction among compound microbial preparations. Wherein, the nitrogen fixing series fixes nitrogen molecules in the nature to make it a nitrogen source for manufacturing fertilizers; the phosphoric acid releasing series unlocks and converts insolvable phosphates in the soil into phosphor, ferrous, and calcium fertilizers; the yeast group series makes it available in the making of vitamins and growing hormones, and decomposes organics to improve disease-resistant sufficiency of the plants; the photosynthetic bacteria series while being applied in manufacturing of glucose secrets carotenoid and eliminates toxic substances including hydrogen sulfide and ammonia; the actinomyces series secrets antibiotic substances at a constant amount on long-term bases to inhibit diseases; and the growing factors producing series also releases on long-term basic a given amount of growing hormones to promote roots, stalks and leaves of crops or plants to grow strong. In some embodiments, one or more of the above described series of microbials are used.

In the course of cross cultivation, each of those eight microbial series maintains intrigue symbiosis and shared prosperity among one another by playing a critical role with secretions of its own particular active organics. For example, the nitrogen fixing series converts the molecular nitrogen into ammoniac nitrogen and the resultant ammoniac nitrogen is partially to be consumed by the nitrogen fixing series, the remaining ammoniac nitrogen is synthesized into organic nitrogen to be consumed by other bacterial series; and the yeast group series may catalyze polysaccharide into simple sugar including glucose to be consumed by lactobacillus to convert into alcohol. Centering on the photosynthetic bacteria series and the yeast group series as leading cores, each microbial series supports activities of other microbial series with its synthetic proficiency while taking advantage of those substances produced by other microbial series to constitute a commonwealth circle. However, behind the big chain of food that relies upon symbiosis substances, a survival game of gigantic resistance and wipe out takes place among one another due to different properties. In the environment seeing violent stimulation, new endocrines are produced. What's more important is that any strain of bacteria survived is practically the top selected one with reliable activities.

Depending on the locality, season, depth of soil, the present invention produces the proper strains of the microbial series. Those who are familiar with the art may apply on various series, e.g. coccus, bacillus, vibrio, or spirillum; different demands of oxygen, e.g., aerobic and/or anaerobic; different environmental requirements, e.g., acidophilus, alkalophilus, psycho-, meso-, or thermophilic to come up with a locality-specific compound microbial preparation and different microbial series may be used to produce compound microbial preparations in various applications, e.g., for fertilizer, pesticide, or promotion growth of flowers and fruits.

Spores and/or colonies that enrich soils and/or provide plant biological control agents are employed in some embodiments. These include bacteria such as Bacillus species, e.g., Bacillus subtilis, Bacillus cereus, Bacillus penetrans, Bacillus licheniformis, and Bacillus megaterium; fungi such as Trichoderma, e.g., Trichoderma hamatum, Trichoderma harzianum, Trichoderma polysporum, Trichoderma konigii, Trichoderma viride; yeast such as Saccharomyces cerevisiae; and mixtures of these. Other examples are given hereafter.

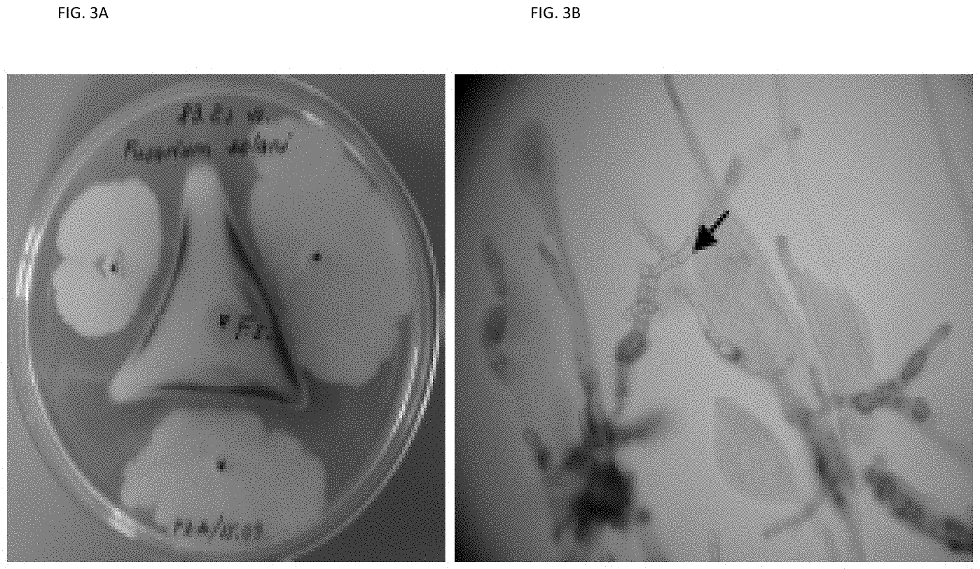

FIG. 3 shows exemplary antifungal activity express by different Bacillus spp. Strains. FIG. 3A shows exemplary Bacillus spp. antagonistic activity against Fusarium solani; while FIG. 3B shows exemplary fungal cell wall degradation, cell lysis and cytoplasm bleeding due to Bacillus spp. extracellular enzymes.

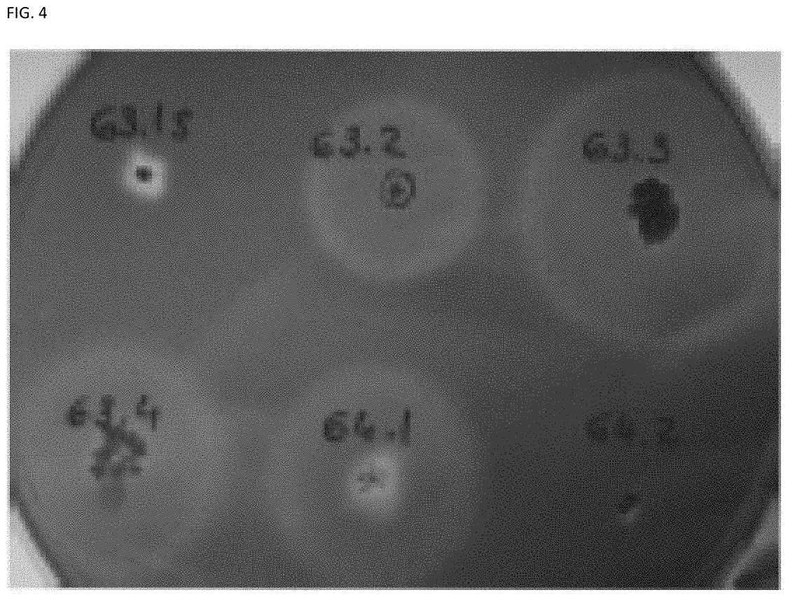

FIG. 4 shows exemplary cellulolytic enzymes synthesized by the biological control agent which can be involved in two plant defense mechanism against phyto-pathogenic fungi. Exemplary cellulase activity exposed on Luria Bertani medium supplement with carboxyl-methyl cellulose, reveal a clear halo of CMC degradation, after two days of Bacillus spp. strains incubation.

In one embodiment called AGN, a natural microbial soil rejuvenation and enrichment provides microbials including enzymes, metabolites and beneficial microbial biomass that aid in building soil structure. In this embodiment, the concentration of microbes can include the following:

| | |
|---|---|
| Bacillus amyloliquefaciens | 7.5 × 10\hat{ }8 cfu/ml |
| Bacillus lichniformis | 7.5 × 10\hat{ }7 cfu/ml |
| Bacillus pumilus | 2.5 × 10\hat{ }8 cfu/ml |
| Bacillus subtilis | 1 × 10\hat{ }8 cfu/ml |
| Bacillus megaterium | 2.5 × 10\hat{ }8 cfu/ml |

The colony-forming unit (CFU or cfu) is a measure of viable bacterial or fungal cells. CFU measures only viable cells. For convenience the results are given as CFU/mL (colony-forming units per milliliter) for liquids, and CFU/g (colony-forming units per gram) for solids.

Humic Acid can be humic substances or leonardite and water. Humic Acid provides the necessary amino acids and protein to support an active microbial population to support active and healthy plant growth.

To deploy, field persons simply dispense the blend alone or mix the blend with dry fertilizer and apply directly to soil as a pre-plant, post-plant or seasonal treatment. The solution can be applied to soil, seeds, and plants. In some embodiments, the solution is not mixed with any other fertilizers or fungicides and deployment of such chemicals should wait at least 72 hours before or after treatment.

The application of the blend creates superior root systems which can efficiently assimilate nutrients and micronutrients in the soil, resulting in higher yields and better plant health for all types of plants, crops, and trees and increases yields, soil-root-plant health, balance soil nutrients, penetrate and loosen clay soils, leach salts from root zones, reduce harmful nematodes, increase nutrient and micronutrient uptake as well as increase cathode ion transfer.

Any microbial spores and/or colonies can be preserved using methods and solutions of some embodiments. Spores and/or colonies of beneficial soil and plant pathogen biological control microorganisms are preferred. Microorganisms that grow rapidly and colonize substrata in soil after treatment with compositions of the invention are particularly preferred. These include, but are not limited to bacteria, e.g., Bacillus species such as Bacillus subtilis, Bacillus cereus, Bacillus penetrans, Bacillus licheniformis, and Bacillus megaterium; fungi, e.g., Trichoderma species such as Trichoderma hamatum, Trichoderma harzianum, Trichoderma polysporum, Trichoderma konigii, and Trichoderma viride; and yeast species such as Saccharomyces cerevisiae. As illustrated below, mixtures of microorganisms can also be preserved, and are preferred in many embodiments. Examples are given hereafter.

In the practice of the system, spores or whole microorganisms, including harvested and/or lyophilized microbial colonies containing spores, are added to solutions. The solutions can be formulated for any use requiring viable microbial spores and/or colonies such as for fertilizers, composting, food products, and pharmaceutical compositions. Liquid fertilizers are preferred for soil enrichment purposes. Water miscible dry powders and/or granules such as lyophilized preparations of spores and/or colonies are preferred in many embodiments. The amount of spores or microorganisms added to solutions of the invention is not fixed per se, and necessarily is dependent upon the degree of soil and/or plant remediation required, the number and identity of microorganism species needed in the formulation, and the concentration of other ingredients in the formulation. Preferred embodiments employ spores and/or colonies in amounts effective to achieve recolonization of the soil by spray application of the composition. Typical embodiments contain sufficient spores and/or colonies to deliver from about 1000 to about 1,000,000 colony forming units (CFU) per square foot when the preparation is delivered.

Microorganisms and/or their spores which can be preserved using formulations of the invention further exhibit a number of desirable characteristics related to soil enrichment and improvement of soil quality described above, such as biological control of plant pathogens (already mentioned); enhancement and/or production of desirable phtyohormones, e.g., auxins, giberillins and cytokinins; and solubilization of phosphates. Certain strains of Bacillus subtilis, for example, inhibit N. Galligena that colonize apple branch scars if applied to trees after leaf fall. E. herbicola and Pseudomonas isolates have been shown to partially control fire blight of pome fruit trees. Several Bacillus species produce antibiotics useful when sprayed as a leaf or needle application on tobacco, Douglas fir, and apple trees, and the natural protection of leaves provided by the buffering capacity of phylloplane microorganisms has been demonstrated. Azobacter, Rhizobium, Bacillus, Klebsiella, Azospirillium, Enterobacter, Serratia, Agrobacterium, Arthrobacter, Aerobacter, Actinomyces, Bacillus, Pseudomonas, and other bacteria stimulate growth, increase yield, and produce other positive results by various mechanisms including enhancing nutrient uptake, increasing germination, enhancing seedling emergence, stimulating de novo biosynthesis, and the like, when applied to fields of various food plants.

The resulting solutions supply carbon-rich organic materials in a bioavailable form for soils and plants together with nutrients that feed the microorganisms as they multiply after application. Solutions of some embodiments provide an excellent food source for the germination of spores and/or colonies when the solutions are applied to soil or water. It is a further advantage that preferred solutions contain a wide variety of naturally occurring metabolites that can be readily absorbed by the growing microorganisms and enhance seed germination, root development, and growth of plants in the soil.

As summarized above, some embodiments are formulated with microorganism spores and/or cultures useful in the prevention, control and/or cure of plant diseases, particularly those of fungal origin. Illustrative examples are provided hereafter. One embodiment, for example, maintains the viability of Bacillus subtilis GB03 (EPA Reg. No. 7501-144), a bacteria recognized to colonize developing root systems, suppressing disease organisms such as Fusarium, Rhizoctonia, Alternaria and Aspergillus that attack root systems. Compositions of the invention can be used to treat developed root systems as well as developing root systems. As the root system develops, grows, and functions, the bacteria grow with the roots, extending protection throughout the growing season. As a result of this biological protection, a vigorous root system can be established and maintained by the plants.

In addition, B. subtilis GB03 has been shown to increase the amount of nodulation by nitrogen-fixing bacteria when used on many legumes. This improvement in nodulation is a result of a healthier root system, allowing more sites for nodules to form from naturally-occurring soil borne nitrogen-fixing bacteria. Illustrative examples follow.

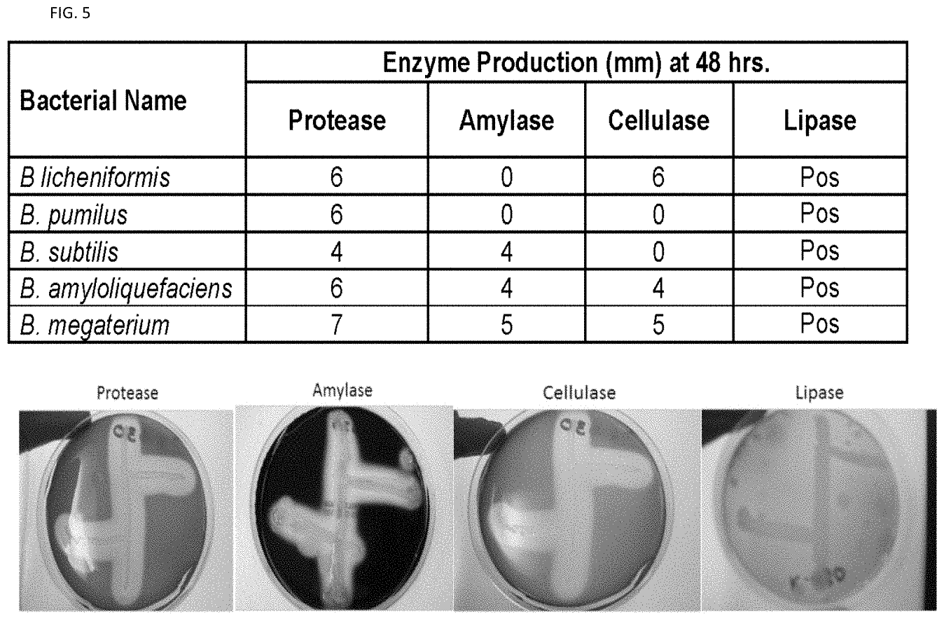

FIG. 5 shows an exemplary AGN enzyme profiles isolation standard. Soil bacteria in the genus Bacillus are well known for contributions to improving soil structure, nutrient availability and as a competitive excluder to harmful pathogens. Bacillus lichniformis produces a variety of extracellular enzymes that are associated with the cycling of nutrients in nature, thus improve nutrient availability and nutrient uptake. Bacillus pumilus is an agricultural fungicide. Growth of the bacterium on plant roots prevents Rhizoctonia and Fusarium spores from germinating. These strains are heavily involved with inhibition of opportunistic pathogens as well as improving nutrient availability and nutrient uptake. Bacillus subtilis does nitrogen fixing; produce inhibitory compounds that reduce the growth of harmful microorganism. It interfere with the germination of plant pathogen spores and their attachment to host plants, acts as a prebiotic conditioning plants own defense mechanisms prior to attack from potential pathogens. Bacillus amyloliquefaciens had anti fungal properties and help nitrogen fixing availability. Bacillus megaterium is a plant growth-promoting rhizobacteria (PGPR) and phosphate solubilizing. It promotes the activation of plant defense responses and secretion of plant growth-regulating substances such as auxins, cytokinins and bacterial volatiles. Phytohormones are involved in the control of growth and in almost every important developmental process in plants. Bacterial secretion of phytohormones can impact root architecture by overproduction of root hairs and lateral roots and subsequently increased nutrient and water uptake, thus contributing to growth.

### Example 1 (AGN LTE)

### Microbes:

| | |
|---|---|
| Bacillus amyloliquefaciens | 7.5 × 10\hat{ }8 cfu/ml |
| Bacillus lichniformis | 7.5 × 10\hat{ }7 cfu/ml |
| Bacillus pumilus | 2.5 × 10\hat{ }8 cfu/ml |
| Bacillus subtilis | 1 × 10\hat{ }8 cfu/ml |
| Bacillus megaterium | 2.5 × 10\hat{ }8 cfu/ml |

### Humic Substances or Leonardite

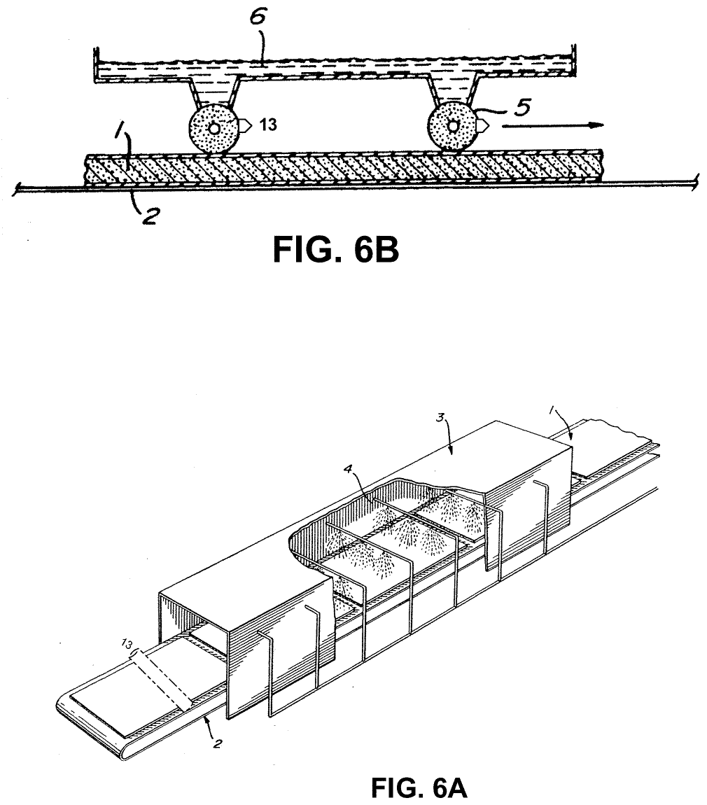

FIGS. 6A-6B show exemplary systems for dry spraying microbes onto humic substances to form granular blend(s). In one embodiment shown in FIG. 6A, urea, kelp or NPK granular blend(s) on a container or board 1 which is disposed on a moving conveyor belt 2. The board 1 enters a spray chamber 3 inside of which a uniform amount of microbes 4 strikes the surfaces of the urea, kelp or NPK granular blends. The rate of liquid sprayed is less than the rate of absorption into the board to avoid liquid accumulation on the surface. The amount of microbes impregnated on the urea, kelp or NPK on board depends on the belt speed or on the spray chamber length. A roller 11 with a plurality of predetermined shapes 13 (or pattern 13) is positioned at the output end, and the roller is positioned by a computer to deposit the microbes in one embodiment. Other manners of microbe impregnation (hopper gun, trowel, or roller) are contemplated. For example, in FIG. 6B the sprayers are replaced by successive porous rolls 5 continuously fed with microbes 6. In other embodiment, a spray gun shown can be conventional Pattern Piston such as that sold by Goldblatt. The spray gun has an air pressure inlet, a spring biased control trigger, a material cavity receiving microbes by gravity flow, a rubber jacket with a hollow beveled head is movable against a rubber washer whereby as the trigger is pulled back, compressed air forces the material through an aperture in the rubber washer. Control variables can be used to affect the microbe coating of the urea, kelp or NPK material being sprayed: the size of the orifice, the liquid state of the material(s) and the air pressure.

The above description is for the purpose of illustrating and not limiting the present invention, and teaching the person of ordinary skill in the art how to practice the invention. It is not intended to detail all those obvious modifications and variations of it which will become apparent to the skilled worker upon reading the description. It is intended, however, that all such obvious modifications and variations be included within the scope of the present invention as defined in the appended claims. The claims are meant to cover the claimed components and steps in any sequence which is effective to meet the objectives there intended, unless the context specifically indicates the contrary.

The patents, papers, and book excerpts cited above are hereby incorporated herein by reference in their entireties.

What is claimed is:

1. A method for forming soil enhancement, comprising:
through a series of breeding cycles guided by genetic testing of microbes, forming a predetermined microbial population with predetermined characteristics in a concentrated form of at least 1×10⁷ cfu/ml (colony-forming units per milliliter); and
dry forming the microbes onto a granular blend of soil amendment.

2. The method of claim 1, comprising selecting a member of Bacillus as the microbe and providing a carrier from one of: liquid, water, wherein the growth medium comprises a carbon source, sugar, molasses, or maltodextrin.

3. The method of claim 1, wherein the granular blend further comprises one of: urea, kelp, seaweed.

4. The method of claim 1, wherein the granular blend further comprises waste.

5. The method of claim 1, wherein the waste comprises one of: fish meal, compost, or manure.

6. The method of claim 1, wherein the waste comprises mammal manure, animal manure, bird manure, chicken manure.

7. The method of claim 1, wherein the granular blend further comprises one or more of: nitrogen, phosphorous, and potassium (NPK).

8. The method of claim 1, comprising selecting the microbe from Bacillus (B.) acidiceler, B. acidicola, B. acidiproducens, B. acidocaldarius, B. acidoterrestrisr, B. aeolius, B. aerius, B. aerophilus, B. agaradhaerens, B. agri, B. aidingensis, B. akibai, B. alcalophilus, B. algicola, B. alginolyticus, B. alkalidiazotrophicus, B. alkalinitrilicus, B. alkalisediminis, B. alkalitelluris, B. altitudinis, B. alveayuensis, B. alvei, B. amyloliquefaciens, B. a. subsp. amyloliquefaciens, B. a. subsp. plantarum, B. amylolyticus, B. andreesenii, B. aneurinilyticus, B. anthracis, B. aquimaris, B. arenosi, B. arseniciselenatis, B. arsenicus, B. aurantiacus, B. arvi, B. aryabhattai, B. asahii, B. atrophaeus, B. axarquiensis, B. azotofixans, B. azotoformans, B. badius, B. barbaricus, B. bataviensis, B. beijingensis, B. benzoevorans, B. beringensis, B. berkeleyi, B. beveridgei, B. bogoriensis, B. boroniphilus, B. borstelensis, B. brevis Migula, B. butanolivorans, B. canaveralius, B. carboniphilus, B. cecembensis, B. cellulosilyticus, B. centrosporus, B. cereus, B. chagannorensis, B. chitinolyticus, B. chondroitinus, B. choshinensis, B. chungangensis, B. cibi, B. circulans, B. clarkii, B. clausii, B. coagulans, B. coahuilensis, B. cohnii, B. composti, B. curdlanolyticus, B. cycloheptanicus, B. cytotoxicus, B. daliensis, B. decisifrondis, B. decolorationis, B. deserti, B. dipsosauri, B. drentensis, B. edaphicus, B. ehimensis, B. eiseniae, B. enclensis, B. endophyticus, B. endoradicis, B. farraginis, B. fastidiosus, B. fengqiuensis, B. firmus, B. flexus, B. foraminis, B. fordii, B. formosus, B. fortis, B. fumarioli, B. funiculus, B. fusiformis, B. galactophilus, B. galactosidilyticus, B. galliciensis, B. gelatini, B. gibsonii, B. ginsengi, B. ginsengihumi, B. ginsengisoli, B. globisporus, B. g. subsp. globisporus, B. g. subsp. marinus, B. glucanolyticus, B. gordonae, B. gottheilii, B. graminis, B. halmapalus, B. haloalkaliphilus, B. halochares, B. halodenitrificans, B. halodurans, B. halophilus, B. halosaccharovorans, B. hemicellulosilyticus, B. hemicentroti, B. herbersteinensis, B. horikoshii, B. horneckiae, B. horti, B. huizhouensis, B. humi, B. hwajinpoensis, B. idriensis, B. indicus, B. infantis, B. infernus, B. insolitus, B. invictae, B. iranensis, B. isabeliae, B. isronensis, B. jeotgali, B. kaustophilus, B. kobensis, B. kochii, B. kokeshiiformis, B. koreensis, B. korlensis, B. kribbensis, B. krulwichiae, B. laevolacticus, B. larvae, B. laterosporus, B. lautus, B. lehensis, B. lentimorbus, B. lentus, B. licheniformis, B. ligniniphilus, B. litoralis, B. locisalis, B. luciferensis, B. luteolus, B. luteus, B. macauensis, B. macerans, B. macquariensis, B. macyae, B. malacitensis, B. mannanilyticus, B. marisflavi, B. marismortui, B. marmarensis, B. massiliensis, B. megaterium, B. mesonae, B. methanolicus, B. methylotrophicus, B. migulanus, B. mojavensis, B. mucilaginosus, B. muralis, B. murimartini, B. mycoides, B. naganoensis, B. nanhaiensis, B. nanhaiisediminis, B. nealsonii, B. neidei, B. neizhouensis, B. niabensis, B. niacini, B. novalis, B. oceanisediminis, B. odysseyi, B. okhensis, B. okuhidensis, B. oleronius, B. oryzaecorticis, B. oshimensis, B. pabuli, B. pakistanensis, B. pallidus, B. pallidus, B. panacisoli, B. panaciterrae, B. pantothenticus, B. parabrevis, B. paraflexus, B. pasteurii, B. patagoniensis, B. peoriae, B. persepolensis, B. persicus, B. pervagus, B. plakortidis, B. pocheonensis, B. polygoni, B. polymyxa, B. popilliae, B. pseudalcalophilus, B. pseudofirmus, B. pseudomycoides, B. psychrodurans, B. psychrophilus, B. psychrosaccharolyticus, B. psychrotolerans, B. pulvifaciens, B. pumilus, B. purgationiresistens, B. pycnus, B. qingdaonensis, B. qingshengii, B. reuszeri, B. rhizosphaerae, B. rigui, B. ruris, B. safensis, B. salarius, B. salexigens, B. saliphilus, B. schlegelii, B. sediminis, B. selenatarsenatis, B. selenitireducens, B. seohaeanensis, B. shacheensis, B. shackletonii, B. siamensis, B. silvestris, B. simplex, B. siralis, B. smithii, B. soli, B. solimangrovi, B. solisalsi, B. songklensis, B. sonorensis, B. sphaericus, B. sporothermodurans, B. stearothermophilus, B. stratosphericus, B. subterraneus, B. subtilis, B. s. subsp. inaquosorum, B. s. subsp. spizizenii, B. s. subsp. subtilis, B. taeanensis, B. tequilensis, B. thermantarcticus, B. thermoaerophilus, B. thermoamylovorans, B. thermocatenulatus, B. thermocloacae, B. thermocopriae, B. thermodenitrificans, B. thermoglucosidasius, B. thermolactis, B. thermoleovorans, B. thermophilus, B. thermoruber, B. thermosphaericus, B. thiaminolyticus, B. thioparans, B. thuringiensis, B. tianshenii, B. trypoxylicola, B. tusciae, B. validus, B. vallismortis, B. vedderi, B. velezensis, B. vietnamensis, B. vireti, B. vulcani, B. wakoensis, B. weihenstephanensis, B. xiamenensis, B. xiaoxiensis, and B. zhanjiangensis.

9. The method of claim 1, wherein the microbes comprise one of: Bacillus amyloliquefaciens at 7.5×10\hat{ }8 cfu/ml, Bacillus lichniformis at 7.5×10\hat{ }7 cfu/ml, Bacillus pumilus at 2.5×10\hat{ }8 cfu/ml, Bacillus subtilis at 1×10\hat{ }8 cfu/ml, Bacillus megaterium at 2.5×10\hat{ }8 cfu/ml.

10. The method of claim 1, wherein the dry forming comprises either dry-spraying or freeze drying with lyophilization.

11. The method of claim 1, comprising iteratively and selectively breed generations of microbes through mutation to arrive at a predetermined microbial solution with screened traits including predetermined microbial genetic profiles and sub-profiles.

12. The method of claim 1, comprising separately cultivating and cross-cultivating multiple microbial series in a specific sequence.

13. The method of claim 12, comprising producing the crossly cultivated microbial series comprises a highly concentrated solution of microbes.

14. The method of claim 1, comprising:
iteratively and selectively breed generations of microbes through mutation to arrive at a predetermined microbial solution with screened traits including predetermined microbial genetic profiles and sub-profiles; and
separately cultivating and cross-cultivating multiple microbial series in a specific sequence.

15. The method of claim 14, comprising producing the crossly cultivated microbial series comprises a highly concentrated solution of microbes.

16. The method of claim 1, wherein the soil amendment comprises humic acid.

17. The method of claim 1, wherein the soil amendment comprises a fertilizer.

18. The method of claim 1, wherein the soil amendment comprises phosphorus.

19. The method of claim 1, wherein the soil amendment comprises nitrogen.

20. The method of claim 1, wherein the soil amendment comprises potassium.
